(19) United States
(12) Patent Application Publication
(10) Pub. No.: US 2025/0289729 A1
(43) Pub. Date: Sep. 18, 2025

(54) BICONICAL TUNGSTEN (MOLYBDENUM) TRIOXIDE POWDER, AND PREPARATION METHOD AND USE THEREOF

(71) Applicant: Zhengzhou University of Light Industry, Zhengzhou (CN)

(72) Inventors: Shizhong WEI, Zhengzhou (CN); Feilong GONG, Zhengzhou (CN); Zexin WEI, Zhengzhou (CN); Tiejun WANG, Zhengzhou (CN); Xiran WANG, Zhengzhou (CN); Ning XIONG, Zhengzhou (CN); Lu YANG, Zhengzhou (CN); Yonghui ZHANG, Zhengzhou (CN)

(21) Appl. No.: 18/779,270

(22) Filed: Jul. 22, 2024

(30) Foreign Application Priority Data
Mar. 12, 2024 (CN) 202410278697.2

(51) Int. Cl. C01G 41/02; C01G 39/02; G01N 33/00

(52) U.S. Cl. CPC C01G 41/02; C01G 39/02; G01N 33/0027; C01P 2002/72; C01P 2004/03; C01P 2004/04; C01P 2004/42

(57) ABSTRACT

A biconical tungsten (molybdenum) trioxide powder, and a preparation method and use thereof are provided. By adjusting the concentrations of glycerol and oxalic acid, the viscosity of the reaction solution could be adjusted, and then the growth of a (001) crystal plane and a (110) crystal plane of the tungsten (molybdenum) trioxide powder could be inhibited; thus, a monodisperse biconical tungsten (molybdenum) trioxide powder could be controllably prepared with a highly exposed (100) crystal plane.

### CROSS REFERENCE TO RELATED APPLICATION

This patent application claims the benefit and priority of Chinese Patent Application No. 202410278697.2 filed with the China National Intellectual Property Administration on Mar. 12, 2024, the disclosure of which is incorporated by reference herein in its entirety as part of the present application.

### TECHNICAL FIELD

The present disclosure relates to the technical field of inorganic materials, and specifically relates to a biconical tungsten (molybdenum) trioxide powder, and a preparation method and use thereof.

### BACKGROUND

Hydrogen is widely used in power generation, chemical industry, food, medical and other fields due to high energy density and pollution-free reaction products. However, the hydrogen is colorless, odorless, and easy to leak, with an explosion limit of 4 vol % to 75 vol % in air. Therefore, it is crucial to develop an efficient and practical method for online and real-time trace detection of hydrogen. Metal oxide semiconductor (MOS) sensors are considered to be ideal candidates for monitoring hydrogen due to their low cost, long life, low power consumption, fast response, and reliable sensitivity. A principle of the MOS sensor is that a sensitive material of the sensor contacts and reacts with gas molecules in the environment, and converts physical and chemical changes on a surface of the sensitive material into electrical signals.

Tungsten (molybdenum) oxide has been proven to be one of the most attractive sensing materials due to structural flexibility, chemical stability, and high oxygen vacancy diffusion coefficient. However, the tungsten (molybdenum) oxide as a sensitive material for the MOS sensors generally shows certain disadvantages, such as slow detection response, i.e., a certain time is required to respond to changes in the target gas, and weak detection response to low-concentration gases. Accordingly, noble metal doping or morphology control is always conducted to improve the above shortcomings to a certain extent. Chinese patent (CN116375091A) reports that needle-like nano-sized tungsten oxide with a controllable appearance is synthesized by adding a control substance dropwise at a controlled dosage in the preparation process. Chinese patent (CN116893205A) reports that a micro-electro-mechanical system (MEMS)-based hydrogen sensor prepared from Au-doped SnO₂ nanoparticles shows a response value of 94 to 150 ppm hydrogen. Chinese patent (CN116794116A) reports that a metal oxide film-type hydrogen sensor is prepared by combination of a traditional MOS with a mesoporous inorganic-organic hybrid layer, which shows a response value of 247.06 and a response time of 1,000 s to 30 ppm H₂ at 75° C., and a response value of 112.54 and a response time of 151 s at 125° C.

The above patents provide ideas for the design and production of hydrogen sensors, but there are still two main problems: 1) although tungsten (molybdenum) trioxide sensitive film materials with different appearances can currently be prepared, it is still challenging to controllably construct semiconductor materials with specific crystal planes; 2) traditional gas sensors have a certain sensitivity to hydrogen, but their detection limits, sensitivity, and response times still cannot meet the safety requirements for rapid and highly sensitive detection of trace hydrogen. As a result, there is an urgent need to develop novel controllable synthesis technologies for gas-sensitive sensing materials to precisely regulate the microstructure of MOS-based gas-sensitive sensing materials, thereby improving their gas-sensing performance.

### SUMMARY

In order to solve the above problems, an object of the present disclosure is to provide a biconical tungsten (molybdenum) trioxide powder, and a preparation method and use thereof.

To achieve the above object, the present disclosure provides a method for preparing the biconical tungsten trioxide powder, including the following steps:
(1) preparing a solution of a soluble tungsten salt with water as a solvent, preparing a solution of a cationic surfactant with diethyl carbinol as a solvent, and adding the solution of the soluble tungsten salt dropwise into the solution of the cationic surfactant under stirring to obtain a mixed solution A;
(2) adding a glycerol solution and an oxalic acid solution dropwise into the mixed solution A in sequence under stirring to obtain a mixed solution B; and
(3) subjecting the mixed solution B to hydrothermal reaction, and subjecting a resulting product to filtering, washing, and drying to obtain the biconical tungsten trioxide powder.

In some embodiments, in step (1), the soluble tungsten salt is one or more selected from the group consisting of ammonium tungstate, sodium tungstate, and potassium tungstate, and the solution of the soluble tungsten salt has a concentration of 0.12 mol/L to 0.86 mol/L.

In some embodiments, in step (1), the diethyl carbinol has a volume of 100 mL to 400 mL, and a volume ratio of the water to the diethyl carbinol is in a range of less than or equal to 1:1.

In some embodiments, in step (1), the cationic surfactant is 1-aminoethyl-2-undecyl imidazoline hydrochloride, and the cationic surfactant has a concentration of 8 mol/L to 35 mol/L.

In some embodiments, in step (1), the stirring is conducted at a speed of 60 r/min to 180 r/min for 3 h to 8 h.

In some embodiments, in step (2), the glycerol solution has a concentration of 5 mol/L to 20 mol/L and a volume of 10 mL to 100 mL; the oxalic acid solution has a concentration of 0.3 mol/L to 1.7 mol/L and a volume of 1 mL to 6 mL; a solvent for the glycerol solution or the oxalic acid solution is deionized water.

In some embodiments, in step (2), the stirring is conducted at a speed of 50 r/min to 200 r/min for 2 h to 6 h.

In some embodiments, in step (3), the hydrothermal reaction is conducted the following two stages: heating the mixed solution B from room temperature to a first-stage reaction temperature of 120° C. to 160° C., and reacting at the first-stage reaction temperature for 2 h to 6 h; and heating a resulting system from the first-stage reaction temperature to a second-stage reaction temperature of 160° C. to 200° C., and reacting at the second-stage reaction temperature for 12 h to 24 h.

In some embodiments, in step (3), heating the mixed solution B from the room temperature to the first-stage reaction temperature is conducted at a heating rate of 8° C./min to 20° C./min, and heating the resulting system from the first-stage reaction temperature to the second-stage reaction temperature is conducted at a heating rate of 0.2° C./min to 1° C./min.

In some embodiments, in step (3), the drying is conducted at a temperature of 40°° C. to 80° C. for 6 h to 24 h.

Also provided is a method for preparing a biconical molybdenum trioxide powder, which is conducted according to the method for preparing the biconical tungsten trioxide powder described above, except that: in step (1), the solution of the soluble tungsten salt is replaced with a solution of a soluble molybdenum salt, the soluble molybdenum salt is one or more selected from the group consisting of magnesium molybdate, sodium molybdate, and ammonium molybdate, and the solution of the soluble molybdenum salt has a concentration of 0.25 mol/L to 0.95 mol/L; and in step (3), the biconical molybdenum trioxide powder is obtained.

Also provided is a biconical tungsten trioxide powder or a biconical molybdenum trioxide powder prepared by the method described above, where the biconical tungsten trioxide powder or the biconical molybdenum trioxide powder has a structure with a highly exposed (100) crystal plane.

Also provided is use of the biconical tungsten trioxide powder or the biconical molybdenum trioxide powder as a sensitive material in a gas sensor for trace detection of H₂, where the gas sensor shows a response value of 96 and a response time of 3 s at an H₂ concentration of 100 ppb. The gas sensor could be used as a high-performance hydrogen sensor in rapid detection for trace amounts of hydrogen.

Compared with the prior art, the present disclosure has the following beneficial effects:

(1) The present disclosure provides a method for preparing the tungsten (molybdenum) trioxide material. By adjusting the concentrations of glycerol and oxalic acid, the viscosity of the reaction solution could be changed, and then the growth of a (001) crystal plane and a (110) crystal plane of the tungsten (molybdenum) trioxide powder could be inhibited. By controlling the amount of oxalic acid added, a monodisperse biconical tungsten (molybdenum) trioxide powder could be controllably prepared with a highly exposed (100) crystal plane. Compared with spherical and flake-shaped tungsten (molybdenum) trioxide powder, the biconical tungsten (molybdenum) trioxide powder has a higher specific surface area and better chemical reactivity.

(2) The present disclosure also provides use of the biconical tungsten (molybdenum) trioxide powder with a highly exposed (100) crystal plane as a sensitive material in a hydrogen sensor, where the hydrogen sensor shows a high response value (96) and a response time of 3 s to 100 ppb H₂ at 100° C., which exhibits faster response at low concentrations. The hydrogen sensor prepared with the biconical tungsten (molybdenum) trioxide powder is far superior to currently reported hydrogen sensors of a same type, and is expected to promote applications of the hydrogen sensor in hydrogen refueling stations and new energy vehicles.

### BRIEF DESCRIPTION OF THE DRAWINGS

FIG. 1A shows a scanning electron microscopy (SEM) image of the biconical tungsten trioxide powder prepared in Example 1 at a low magnification;

FIG. 1B shows a scanning electron microscopy (SEM) image of the biconical tungsten trioxide powder prepared in Example 1 at a high magnification;

FIG. 2A shows a transmission electron microscopy (TEM) image of the biconical tungsten trioxide powder prepared in Example 2 at a low magnification;

FIG. 2B shows a transmission electron microscopy (TEM) image of the biconical tungsten trioxide powder prepared in Example 2 at a high magnification;

FIG. 3 shows an X-ray diffraction (XRD) pattern of the biconical tungsten trioxide powder prepared in Example 2;

FIG. 4 shows response values of the biconical tungsten trioxide powder prepared in Example 3 at different temperatures;

FIG. 5 shows response values of the biconical tungsten trioxide powder prepared in Example 3 at different concentrations;

FIG. 6 shows a response recovery curve of the biconical tungsten trioxide powder prepared in Example 3; and

FIG. 7 shows a comparison of hydrogen detection values at low concentrations between the biconical tungsten trioxide powder prepared in Example 3 and other materials disclosed in different literatures.

### DETAILED DESCRIPTION OF THE EMBODIMENTS

To order to better understand the content of the present disclosure, the technical solutions of the present disclosure will be further described below with reference to examples. The following examples provide detailed implementation methods and operating steps based on the technical solutions of the present disclosure, but the scope of the present disclosure is not limited to the following examples.

### Example 1

(1) An ammonium tungstate solution with a concentration of 0.25 mol/L was prepared with 80 mL of water as a solvent, a 1-aminoethyl-2-undecyl imidazoline hydrochloride solution with a concentration of 10 mol/L was prepared with 240 mL of diethyl carbinol as a solvent, and the ammonium tungstate solution was added dropwise into the 1-aminoethyl-2-undecyl imidazoline hydrochloride solution by stirring at 80 r/min for 5 h to obtain a mixed solution A.

(2) 20 mL of a glycerol solution with a concentration of 14 mol/L was added dropwise into the mixed solution A under stirring to obtain a mixture, and then 1.5 mL of an oxalic acid solution with a concentration of 0.5 mol/L was added dropwise into the mixture under stirring to obtain a mixed solution B; where the stirring was conducted at 120 r/min for 6 h.

(3) The mixed solution B was subjected to hydrothermal reaction, specifically, the mixed solution B was heated from room temperature to 150° C. at 8° C./min, and reacted at 150°° C. for 4 h, and then a resulting system was heated from 150° C. to 190° C. at 0.5° C./min, and reacted at 190° C. for 20 h. Then, a resulting reaction product was filtered, washed, and dried at 40° C. for 18 h to obtain a biconical tungsten trioxide powder.

### Example 2

(1) A sodium tungstate solution with a concentration of 0.5 mol/L was prepared with 85 mL of water as a solvent, a 1-aminoethyl-2-undecyl imidazoline hydrochloride solution with a concentration of 12 mol/L was prepared with 255 mL of diethyl carbinol as a solvent, and the sodium tungstate solution was added dropwise into the 1-aminoethyl-2-undecyl imidazoline hydrochloride solution by stirring at 100 r/min for 4 h to obtain a mixed solution A.

(2) 12 mL of a glycerol solution with a concentration of 18 mol/L was added dropwise into the mixed solution A under stirring to obtain a mixture, and then 2 mL of an oxalic acid solution with a concentration of 0.5 mol/L was added dropwise into the mixture under stirring to obtain a mixed solution B; where
the stirring was conducted at 160 r/min for 3 h.

(3) The mixed solution B was subjected to hydrothermal reaction, specifically, the mixed solution B was heated from room temperature to 120° C. at 10° C./min, and reacted at 120°° C. for 3 h, and then a resulting system was heated from 120° C. to 200° C. at 1° C./min, and reacted at 200°° C. for 24 h. Then, a resulting reaction product was filtered, washed, and dried at 50° C. for 20 h to obtain a biconical tungsten trioxide powder.

### Example 3

(1) A potassium tungstate solution with a concentration of 0.6 mol/L was prepared with 100 ml of water as a solvent, a 1-aminoethyl-2-undecyl imidazoline hydrochloride solution with a concentration of 20 mol/L was prepared with 300 mL of diethyl carbinol as a solvent, and the potassium tungstate solution was added dropwise into the 1-aminoethyl-2-undecyl imidazoline hydrochloride solution by stirring at 70 r/min for 8 h to obtain a mixed solution A.

(2) 50 mL of a glycerol solution with a concentration of 10 mol/L was added dropwise into the mixed solution A under stirring to obtain a mixture, and then 3.5 mL of an oxalic acid solution with a concentration of 0.6 mol/L was added dropwise into the mixture under stirring to obtain a mixed solution B; where
the stirring was conducted at 180 r/min for 2 h.

(3) The mixed solution B was subjected to hydrothermal reaction, specifically, the mixed solution B was heated from room temperature to 130° C. at 15° C./min, and reacted at 130°° C. for 5 h, and then a resulting system was heated from 130° C. to 200° C. at 0.8° C./min, and reacted at 200°° C. for 21 h. Then, a resulting reaction product was filtered, washed, and dried at 80° C. for 8 h to obtain a biconical tungsten trioxide powder.

### Example 4

(1) A magnesium molybdate solution with a concentration of 0.85 mol/L was prepared with 90 mL of water as a solvent, a 1-aminoethyl-2-undecyl imidazoline hydrochloride solution with a concentration of 32 mol/L was prepared with 270 mL of diethyl carbinol as a solvent, and the magnesium molybdate solution was added dropwise into the 1-aminoethyl-2-undecyl imidazoline hydrochloride solution by stirring at 120 r/min for 3 h to obtain a mixed solution A.

(2) 70 mL of a glycerol solution with a concentration of 8 mol/L was added dropwise into the mixed solution A under stirring to obtain a mixture, and then 2 mL of an oxalic acid solution with a concentration of 1.6 mol/L was added dropwise into the mixture under stirring to obtain a mixed solution B; where
the stirring was conducted at 200 r/min for 2 h.

(3) The mixed solution B was subjected to hydrothermal reaction, specifically, the mixed solution B was heated from room temperature to 140° C. at 9° C./min, and reacted at 140°° C. for 5 h, and then a resulting system was heated from 140° C. to 180° C. at 1° C./min, and reacted at 180° C. for 19 h. Then, a resulting reaction product was filtered, washed, and dried at 55° C. for 19 h to obtain a biconical molybdenum trioxide powder.

### Example 5

(1) An ammonium molybdate solution with a concentration of 0.95 mol/L was prepared with 70 mL of water as a solvent, a 1-aminoethyl-2-undecyl imidazoline hydrochloride solution with a concentration of 30 mol/L was prepared with 210 mL of diethyl carbinol as a solvent, and the ammonium molybdate solution was added dropwise into the 1-aminoethyl-2-undecyl imidazoline hydrochloride solution by stirring at 160 r/min for 4 h to obtain a mixed solution A.

(2) 35 mL of a glycerol solution with a concentration of 13 mol/L was added dropwise into the mixed solution A under stirring to obtain a mixture, and then 6 mL of an oxalic acid solution with a concentration of 0.7 mol/L was added dropwise into the mixture under stirring to obtain a mixed solution B; where
the stirring was conducted at 90 r/min for 6 h.

(3) The mixed solution B was subjected to hydrothermal reaction, specifically, the mixed solution B was heated from room temperature to 120° C. at 18° C./min, and reacted at 120°° C. for 5 h, and then a resulting system was heated from 120° C. to 170° C. at 0.5° C./min, and reacted at 170° C. for 15 h. Then, a resulting reaction product was filtered, washed, and dried at 80° C. for 10 h to obtain a biconical molybdenum trioxide powder.

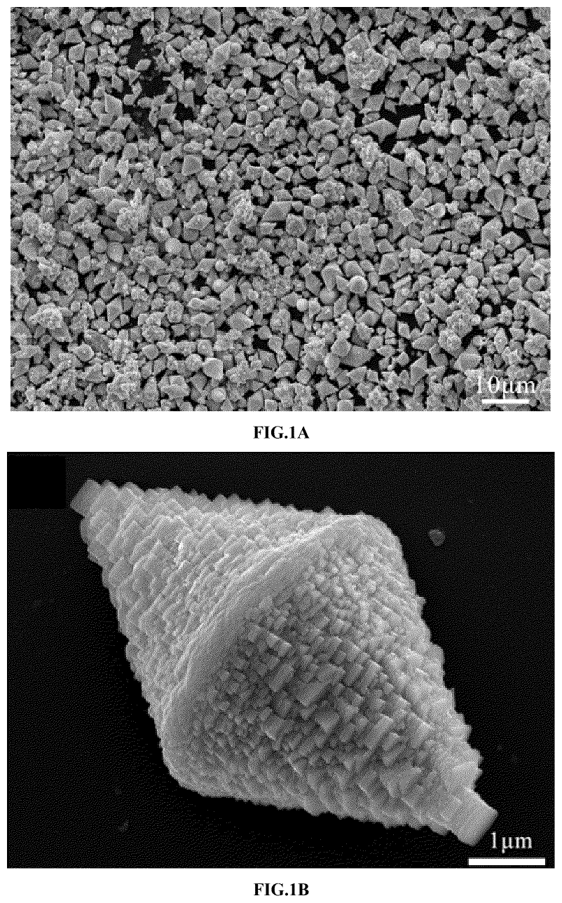

FIG. 1A-FIG. 1B show SEM images of the biconical tungsten trioxide powder prepared in Example 1, where FIG. 1A represents a low-magnification appearance, and FIG. 1B represents a high-magnification appearance. From FIG. 1A-FIG. 1B, it can be seen that the reaction product is biconical.

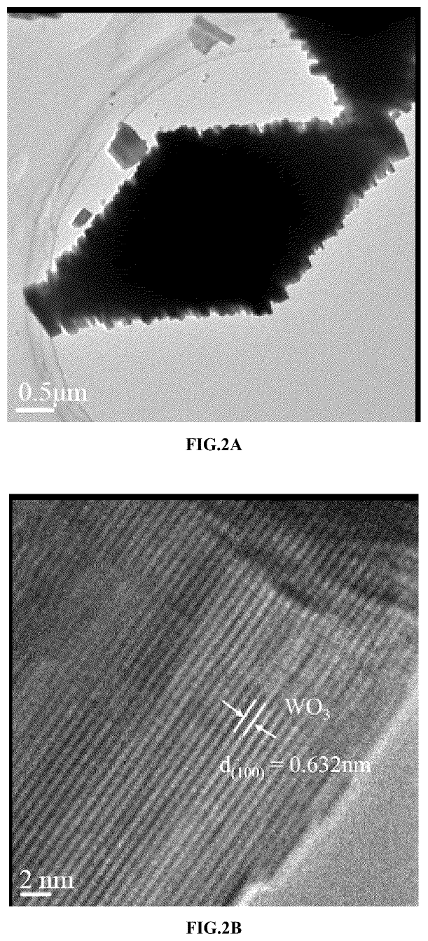

FIG. 2A-FIG. 2B show TEM images of the biconical tungsten trioxide powder prepared in Example 2, where FIG. 2A represents a low-magnification appearance, and FIG. 2B represents a high-magnification appearance. From FIG. 2A-FIG. 2B, it can be seen that, the reaction product is biconical, with a lattice spacing of 0.632 nm, corresponding to the (100) crystal plane of tungsten trioxide.

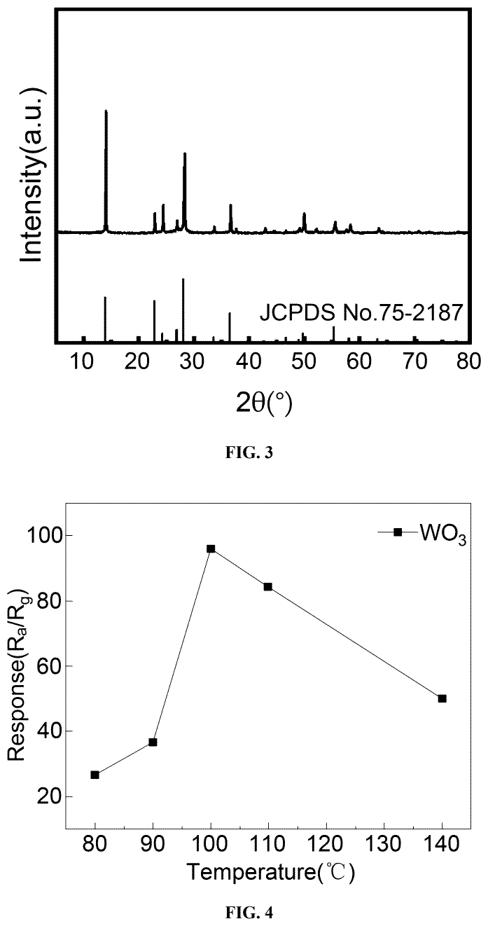

FIG. 3 shows an XRD pattern of the biconical tungsten trioxide powder prepared in Example 2. From FIG. 2, it can be seen that, the results correspond to the card (JCPDS No.75-2187), indicating that the prepared product is tungsten trioxide.

FIG. 4 shows response values of the biconical tungsten trioxide powder prepared in Example 3 at different temperatures. From FIG. 4, it can be seen that the response value is the maximum at 100° C., that is, the biconical tungsten trioxide powder has the best gas-sensing performance at this temperature.

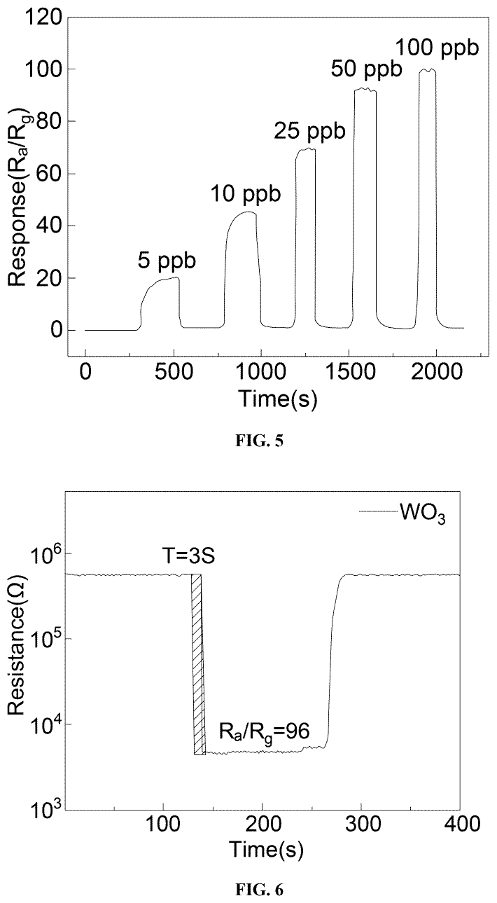

FIG. 5 shows response values of the biconical tungsten trioxide powder prepared in Example 3 at different concentrations. From FIG. 5, it can be seen that the gas sensor prepared with the biconical tungsten trioxide powder as a sensitive material has a response value of up to 96 at 100° C. and a H₂ concentration of 100 ppb.

FIG. 6 shows a response recovery curve of the biconical tungsten trioxide powder prepared in Example 3. From FIG. 6, it can be seen that the biconical tungsten trioxide powder has a response time of 3 s at 100° C. and a H₂ concentration of 100 ppb.

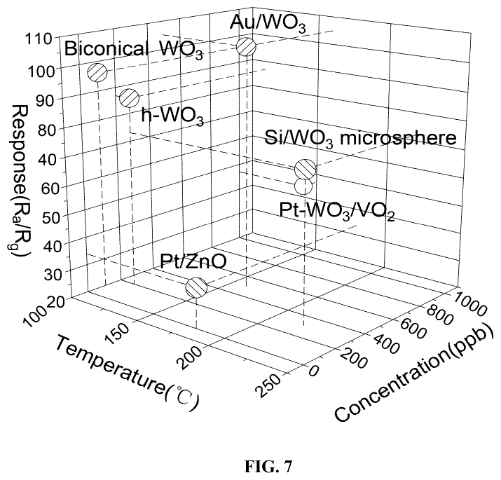

FIG. 7 shows a comparison of hydrogen detection values at low concentrations between the biconical tungsten trioxide powder prepared in Example 3 and other materials disclosed in different literatures. From FIG. 7, it can be seen that the gas sensor prepared with the biconical tungsten trioxide powder as a sensitive material in the present disclosure exhibits an excellent gas sensing performance.

The above are merely preferred embodiments of the present disclosure rather than limitations on the present disclosure in any form. The present disclosure can also have other forms of embodiments based on the above structures and functions, which are not listed one by one. Therefore, any simple modifications, equivalent substitutions, equivalent changes, and modifications made to the above embodiments according to the technical essence of the present disclosure without departing from the contents of the technical solutions of the present disclosure still fall within the scope of the technical solutions of the present disclosure.

What is claimed is:

1. A method for preparing a biconical tungsten trioxide powder, comprising the following steps:
(1) preparing a solution of a soluble tungsten salt with water as a solvent, preparing a solution of a cationic surfactant with diethyl carbinol as a solvent, and adding the solution of the soluble tungsten salt dropwise into the solution of the cationic surfactant under stirring to obtain a mixed solution A;
(2) adding a glycerol solution and an oxalic acid solution dropwise into the mixed solution A in sequence under stirring to obtain a mixed solution B; and
(3) subjecting the mixed solution B to hydrothermal reaction, and subjecting a resulting product to filtering, washing, and drying to obtain the biconical tungsten trioxide powder.

2. The method of claim 1, wherein in step (1), the soluble tungsten salt is one or more selected from the group consisting of ammonium tungstate, sodium tungstate, and potassium tungstate, and the solution of the soluble tungsten salt has a concentration of 0.12 mol/L to 0.86 mol/L.

3. The method of claim 1, wherein in step (1), the diethyl carbinol has a volume of 100 mL to 400 mL, and a volume ratio of the water to the diethyl carbinol is in a range of less than or equal to 1:1.

4. The method of claim 1, wherein in step (1), the cationic surfactant is 1-aminoethyl-2-undecyl imidazoline hydrochloride, and the cationic surfactant has a concentration of 8 mol/L to 35 mol/L; and the stirring is conducted at a speed of 60 r/min to 180 r/min for 3 h to 8 h.

5. The method of claim 1, wherein in step (2),
the glycerol solution has a concentration of 5 mol/L to 20 mol/L and a volume of 10 mL to 100 mL;
the oxalic acid solution has a concentration of 0.3 mol/L to 1.7 mol/L and a volume of 1 mL to 6 mL;
a solvent for the glycerol solution or the oxalic acid solution is deionized water; and
the stirring is conducted at a speed of 50 r/min to 200 r/min for 2 h to 6 h.

6. The method of claim 1, wherein in step (3), the hydrothermal reaction is conducted the following two stages:
heating the mixed solution B from room temperature to a first-stage reaction temperature of 120° C. to 160° C., and reacting at the first-stage reaction temperature for 2 h to 6 h; and
heating a resulting system from the first-stage reaction temperature to a second-stage reaction temperature of 160° C. to 200° C., and reacting at the second-stage reaction temperature for 12 h to 24 h;
and wherein the drying in step (3) is conducted at a temperature of 40° C. to 80° C. for 6 h to 24 h.

7. The method of claim 6, wherein in step (3), heating the mixed solution B from the room temperature to the first-stage reaction temperature is conducted at a heating rate of 8° C./min to 20° C./min, and heating the resulting system from the first-stage reaction temperature to the second-stage reaction temperature is conducted at a heating rate of 0.2° C./min to 1° C./min.

8. A method for preparing a biconical molybdenum trioxide powder, wherein the method for preparing the biconical molybdenum trioxide powder is conducted according to the method of claim 1, except that:
in step (1), the solution of the soluble tungsten salt is replaced with a solution of a soluble molybdenum salt, the soluble molybdenum salt is one or more selected from the group consisting of magnesium molybdate, sodium molybdate, and ammonium molybdate, and the solution of the soluble molybdenum salt has a concentration of 0.25 mol/L to 0.95 mol/L; and
in step (3), the biconical molybdenum trioxide powder is obtained.

9. The method of claim 8, wherein in step (1), the diethyl carbinol has a volume of 100 mL to 400 mL, and a volume ratio of the water to the diethyl carbinol is in a range of less than or equal to 1:1.

10. The method of claim 8, wherein in step (1), the cationic surfactant is 1-aminoethyl-2-undecyl imidazoline hydrochloride, and the cationic surfactant has a concentration of 8 mol/L to 35 mol/L; and the stirring is conducted at a speed of 60 r/min to 180 r/min for 3 h to 8 h.

11. The method of claim 8, wherein in step (2),
the glycerol solution has a concentration of 5 mol/L to 20 mol/L and a volume of 10 mL to 100 mL;
the oxalic acid solution has a concentration of 0.3 mol/L to 1.7 mol/L and a volume of 1 mL to 6 mL;
a solvent for the glycerol solution or the oxalic acid solution is deionized water; and
the stirring is conducted at a speed of 50 r/min to 200 r/min for 2 h to 6 h.

12. The method of claim 8, wherein in step (3), the hydrothermal reaction is conducted by the following two stages:
heating the mixed solution B from room temperature to a first-stage reaction temperature of 120°° C. to 160° C., and reacting at the first-stage reaction temperature for 2 h to 6 h; and
heating a resulting system from the first-stage reaction temperature to a second-stage reaction temperature of 160° C. to 200°° C., and reacting at the second-stage reaction temperature for 12 h to 24 h;
and wherein the drying in step (3) is conducted at a temperature of 40° C. to 80° C. for 6 h to 24 h.

13. The method of claim 12, wherein in step (3), heating the mixed solution B from the room temperature to the first-stage reaction temperature is conducted at a heating rate of 8° C./min to 20° C./min, and heating the resulting system from the first-stage reaction temperature to the second-stage reaction temperature is conducted at a heating rate of 0.2° C./min to 1° C./min.

14. A biconical tungsten trioxide powder prepared by the method of claim 1, wherein the biconical tungsten trioxide powder has a structure with a highly exposed (100) crystal plane.

15. A biconical molybdenum trioxide powder prepared by the method of claim 8, wherein the biconical molybdenum trioxide powder has a structure with a highly exposed (100) crystal plane.

16. A hydrogen sensor, wherein the biconical tungsten trioxide powder of claim 14 is used as a sensitive material in the hydrogen sensor for trace detection of H₂, and the hydrogen sensor shows a response value of 96 and a response time of 3 s at an H₂ concentration of 100 ppb.

17. A hydrogen sensor, wherein the biconical molybdenum trioxide powder of claim 15 is used as a sensitive material in the hydrogen sensor for trace detection of H₂, and the hydrogen sensor shows a response value of 96 and a response time of 3 s at an H₂ concentration of 100 ppb.
